(19) United States
(12) Patent Application Publication
(10) Pub. No.: US 2025/0046456 A1
(43) Pub. Date: Feb. 6, 2025

(54) SYSTEMS AND METHODS FOR SOURCE MODALITY LATENT DOMAIN LEARNING AND FEW-SHOT DOMAIN ADAPTATION

(71) Applicant: BOARD OF REGENTS, THE UNIVERSITY OF TEXAS SYSTEM, Austin, TX (US)

(72) Inventors: Peyman Najafirad, San Antonio, TX (US); Rinu Joseph, San Antonio, TX (US); Arun Das, Pittsburgh, PA (US)

(21) Appl. No.: 18/791,561

(22) Filed: Aug. 1, 2024

Related U.S. Application Data
(60) Provisional application No. 63/530,161, filed on Aug. 1, 2023.

(51) Int. Cl. G16H 50/20; A61B 5/291; G06T 7/00; G06V 10/80

(52) U.S. Cl. CPC G16H 50/20; A61B 5/291; G06T 7/0012; G06V 10/811; G06T 2207/20081; G06T 2207/20084; G06T 2207/30201

(57) ABSTRACT

The present disclosure presents system and methods for obtaining multimodal input data of a subject, wherein the multimodal input data comprises at least two input modalities of data; extracting features from the multimodal input data; learning multimodal signal correlations and source latent distribution alignment from the extracted features of the multimodal input data; training and optimizing a multimodal machine learning algorithm on input labeled data to learn local features of each modality of the multimodal input data; and/or executing, by a computer system, the trained multimodal machine learning algorithm to predict a cognitive state or disorder of the subject using the learned multimodal signal correlations and source latent distribution alignment.

### CROSS-REFERENCE TO RELATED APPLICATION

This application is claims priority to co-pending U.S. provisional application entitled, “System and Methods for Source Modality Latent Domain Learning and Few-Shot Domain Adaptation,” having application Ser. No. 63/530,161, filed Aug. 1, 2023, which is entirely incorporated herein by reference.

### TECHNICAL FIELD

This application relates to the system and methods for using machine learning (ML) algorithms to learn source domain distribution from a large amount of unlabeled data.

### BACKGROUND

Human brain activity is in a constant state of flux. A main goal of cognitive neuroscience is to study the dynamics of the brain and how they relate to behavioral output. Behavior is preceded by information flow in brain and spinal networks that plan and execute a given behavior. Emerging work suggests that voluntary behaviors are usually accompanied by various involuntary micro movements.

Dysfunction of nervous system as well as the spinal cord result in neurological disorders like Alzheimer's, Epilepsy & seizures, Parkinson's disease and speech disfluency. Electroencephalogram (EEG), a neurophysiological measurement which can detect the abnormalities in the neural signals, can be useful in understanding the brain dynamics in neurological diseases and how they differ from normal brain states. Certain neurological disorders, like Alzheimer's and speech disfluency, can cause tremors and some other “secondary behaviors,” such as eye-blinking, jerks in jaws, or other involuntary movements of head or limbs, while speaking. Facial expression analysis from a recorded video can be used to study these involuntary movements which are associated with different emotional states. Similarly, functional Magnetic Resonance Imaging (fMRI), is another diagnostic method to detect the abnormalities inside the brain by capturing the changes in blood flow to brain. By capturing the neuronal activities from the human brain using EEG, researchers can better understand how humans see and think from their facial behavior and emotions.

### BRIEF DESCRIPTION OF THE DRAWINGS

FIG. 1 illustrates an exemplary system architecture for source modality latent domain learning in accordance with various embodiments of the present disclosure.

FIG. 2 illustrates the high-level architecture of source modality latent domain learning (SMDL) framework in accordance with various embodiments of the present disclosure.

FIG. 3 illustrates an exemplary latent domain source distribution alignment (LDSDA) framework in accordance with various embodiments of the present disclosure.

FIG. 4 illustrates an exemplary source modality latent domain learning explainer framework in accordance with various embodiments of the present disclosure.

FIG. 5 illustrates an exemplary the SMDL framework for predicting speech disfluency in an example scenario in accordance with various embodiments of the present disclosure.

FIG. 6 illustrates an exemplary computer system usable with systems and methods of the present disclosure.

### DETAILED DESCRIPTION

The human face exhibits both voluntary and involuntary muscle movements, and analysis of facial movements can be used to assess and diagnose various diseases. A common way to define facial muscle movements is by encoding their activity as facial action unit (AU) patterns. Recent research in Artificial Intelligence (AI) algorithms has shown impressive results in predicting various diseases, emotions, behavior, and much more from both EEG and facial muscle movements using automated algorithms. The successful encoding of facial muscle movement patterns as facial Action Units (AU) based on the Facial Action Unit Coding System (FACS) has shown the ability to quantify human attention, affect, and pain. Relatedly, AI algorithms using EEG signals as inputs can distinguish among cognitive states and are relevant to understanding neurological disorders such as Alzheimer's disease and Parkinson's disease.

However, the scope of examining multiple modalities together has not been explored before. For example, incorporating facial muscle activity and EEG or fMRI into interpretable machine learning models will provide insight into how peripheral measures of microexpressions relate to internal neurocognitive states. Accordingly, the present disclosure relates to the system and methods for source modality latent domain learning (SMDL). With source modality latent domain learning (SMDL), a computer model can make predictions by learning the association between modalities. To give an example, SMDL can learn the temporal dynamics of both EEG or fMRI and facial muscle movements during speech preparation, from a small amount (“few-shot”) of labeled data to predict diseased versus non-diseased cases, and multimodal explainability analysis can be performed to identify the distinct facial expressions and brain regions correlated with the disorder.

Accordingly, in various embodiments of the present disclosure, exemplary systems and methods of the present disclosure utilize neural networks to learn the correlation between multiple modalities by applying signal transformations and training a deep learning model for each modality with a small amount of labeled data to learn the local features of each modality. In various embodiments, a latent representation of modalities is made closer by applying an alignment loss during optimization.

As such, various embodiments of system and methods of the present disclosure use machine learning (ML) algorithms to learn source domain distribution from a large amount of unlabeled data and later transfer the knowledge to train a ML algorithm on a small amount of labeled target domain data. Such systems and methods have applicability to applications in use cases where there is a dearth of labeled multimodal datasets, such as in healthcare.

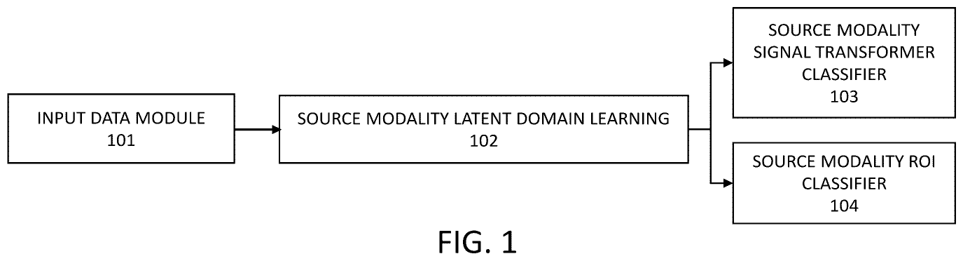

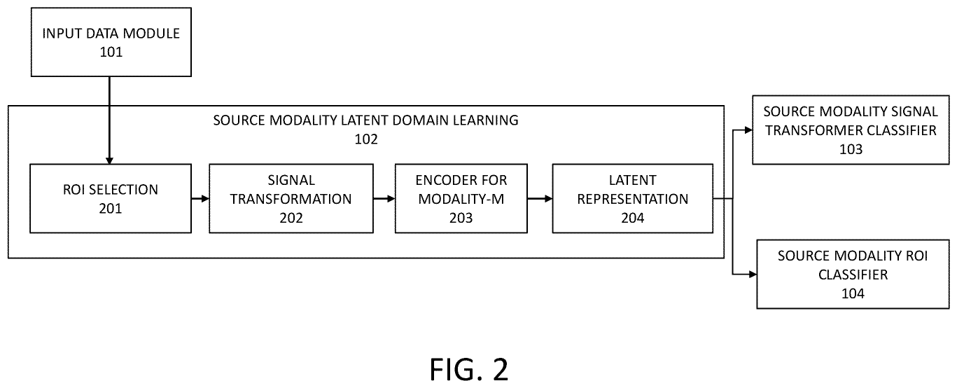

In accordance with various embodiments, an exemplary overall system architecture of the disclosure is illustrated in FIG. 1 and FIG. 2 and includes through the input data module 101, SMDL module 102, source modality signal transformer classifier module 103, and source modality region of interest classifier module 104. Accordingly, in one exemplary embodiment, data is collected in input data module 101 from multiple input sources or modalities (modality of input dataset), such as Electroencephalogram (EEG), camera, mobile phones, or wearable devices. An exemplary SMDL algorithm is then applied to each modality in module 102. During the pre-training phase in SMDL, signal transformations are applied on selected region-of-interest (ROI). A loss function [symbol] ₛₜ(w, θₛₜ), a source modality signal transformer classifier module 103 identifies the correct signal transformation applied to the selected ROI. A source modality ROI classifier module 104 uses another loss function [symbol] _{win}(w) to find the ROI in which the transformation was applied.

In accordance with various embodiments, FIG. 2 illustrates a high-level architecture of an exemplary source modality latent domain learning (SMDL) framework. Here, the SMDL algorithm is applied to data from each input modality 101 of an input dataset to learn temporal correspondences (time synchronization features) between the input features. Here, the SMDL module 102 learns faithful representations from a large amount of unlabeled datasets which are readily available in most domains. Even in healthcare, where there is a scarcity of labeled data, a large amount of unlabeled clinical data available is available. An exemplary SMDL module utilizes a ROI selection module 201, a signal transformation module 202, and a modality encoder 203 of a deep learning based encoded network to learn latent representations of the source modality 204.

In exemplary and non-limiting SDML architecture, three variants (SDML-A, SDML-B, and SDML-C) of network architecture is built for each modality encoder. In SDML-A, a modality 1 encoder H_{M1} contains 4 convolutional (Conv) layers with {16, 32, 64, 64} kernels respectively, all shaped 1×17. This is followed by depth-wise (DepthConv) and separable (SepConv) convolutions. Final embedding Zₘ₁ is of 1×64 dimensions. In a modality 2 encoder, H_{M2}, data extracted from one Conv layer with 16 kernels, all shaped 1×62, is passed to a DepthConv layer with 16 kernels and depth factor 2 to compress the data along the channels. A SepConv layer with 16 kernels is then used to summarize individual feature maps and later flattened to an embedding Z_{M2} of 1×64 dimensions.

In the SDML-B architecture (280 k parameters), both H_{M1} and H_{M2} contain 3 Conv layers with {16, 32, 64} kernels respectively, all shaped 3×3 with max-pooling layers to create embeddings of 1×64 in each path. To study the impact of additional layers, SDML-C (317 k parameters) has an additional Conv layer with 64 kernels of size 3×3 in both branches.

Given that the goal of SMDL is to learn latent representations that will improve the learning process of downstream tasks that are carried out on a trained encoder network 203 and to guarantee the learning of good latent representations that are conducive to further optimizations, an exemplary system/method selects a region of interest from the input data matrix from the input data module 101. Once a ROI is randomly selected, an exemplary system/method applies a signal transformation to the selected ROI. In order to correctly predict what signal transformation was carried out and in which ROI, and exemplary system/method uses cross-entropy loss to tune the parameters of an encoder model 203 to learn temporal dynamics of the input data features using classifiers deployed by modules 103 and 104.

For example, with access to a large amount of facial videos, the encoder network 203 can be taught to faithfully compress the input videos to a dense latent representation which has information about the temporal muscle movements of the individual. Thus, an exemplary SMDL system acts as a compressor, compressing the large input videos to dense latent representations which can then be tuned to predict various downstream tasks.

However, for many real-world applications such as in healthcare, neuroscience, marketing, speech therapy, education and more, the behavioral aspects of decision-making are always multimodal. For example, for speech therapy, one must look at both the functional aspects of brain circuitry using imaging techniques or EEG signal analysis, and also the external facial muscle-movements that are associated with pre-speech and post-speech along with the muscle movements during speech. This kind of analysis requires a collection of input modalities.

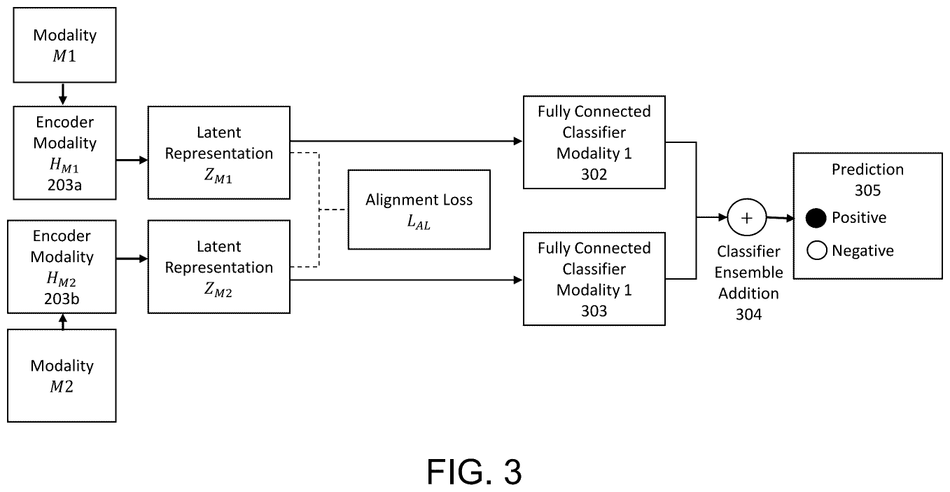

Hence, in FIG. 3, an exemplary Latent Domain Source Distribution Alignment (LDSDA) framework is presented that brings the distribution of the input modality datasets M1, M2 closer to each other while retraining the encoder network 203a, 203b. In particular, retraining the encoder network 203 ensures that the distribution alignment happens successfully, and in SMDL, the latent representations of modality 2 encoder Z_{M2} are made closer to the latent representations of modality 1 encoder Z_{M1} using an alignment loss LAL function during optimization. Relatedly, the now aligned distributions are fed to fully connected classifiers 302, 303 for individual modalities for a specific task. These classifiers 302, 303 provide an output vector each with logit information of the output decision. The information from multiple modalities can be further correlated using a classifier ensemble addition of individual logits via a classifier ensemble addition module 304. The final classifier predictions 306 on whether the multimodal inputs can be classified as a positive or negative outcome, can be thus made from an informed decision using multimodal inputs.

To demonstrate, input signals from modality M1 and modality M2 can be correlated. For example, in speech disfluency studies input signals from EEG X_{EEG} and input frames from facial video X_{AU} containing the individual facial movement patterns for the time t can be examined together to understand the correlation between brain states and involuntary movements associated with voluntary behaviors. However, the dearth of labeled multimodal data in the field of healthcare is a challenge in training an AI model. To address this issue, an exemplary deep learning domain adaptation Source Modality Latent Domain Learning (SMDL) system is configured to learn the domain distribution from unlabeled data by pretext task training and then training the ML algorithm with a small amount of labeled data.

Inputs to multimodal networks often have correlations which also help the network to learn features between the inputs. Therefore, parallel convolutional neural networks (CNNs) H_{M1} and H_{M2} for modality 1 and modality 2 are used to create dense representations of corresponding modality inputs and to learn meaningful representations from both modalities in a combined multimodal training paradigm.

In SMDL, the latent representations of modality 2 encoder Z_{M2} are aimed to be closer to the latent representations of modality 1 Z_{M1} encoder using an alignment loss [symbol] _{AL} function during optimization. The design of modality pretext task is based on existing experimental metadata such that the meaningful representations can be learned. For example, with an EEG pretext task latent representation, a facial encoder Z_{AU} can not only learn about facial microexpressions, but also about the different cognitive contexts.

In the pre-training of H_{M1}, the goal is to predict paradigm information Yₚₐᵣₐ and estimate the loss [symbol] ₚₐᵣₐ using cross entropy. Similarly, during the pre-training of H_{M2}, a signal transformation can be applied on a time window w to improve the learning performance. Two loss functions [symbol] ₛₜ (w, θₛₜ) can be defined to find the correct signal transformation applied to the window w and another loss function [symbol] _{win}(w) can be used to find the window w in which the transformation is applied.

In various embodiments, a weighted ensemble method with non-parametric multipliers δ and γ is used to combine the embeddings Z_{M1} and Z_{M2} and a final prediction 306 is made using the Sigmoid threshold on the weighted classifiers of H_{M1} and H_{M2}. To explain the correlations between the modalities 1 and 2, an explanation map for each modality can be generated. For example, to understand multimodal prediction model f(x_{M1}, x_{M2}) based on two inputs, considering fz as the embedding layer of f, a linear approximation of the combination shapley values for each modality can be calculated based on DeepLIFT multipliers m. An average marginal contribution of features based on local feature attributions of both modalities can be calculated citing the additive nature of Shapley explanations, with feature removals of corresponding inputs X_{M1} and X_{M2} influence ϕᵢ(f_{z}, y). The loss equations and final result can be described as below.

$${ℒ}_{para} = - {\sum }_{para = 1}^{4} {y}_{para} log ( {p}_{para} ) \\ {ℒ}_{st} ( w , {θ}_{st} ) = - log P ( \tilde{st} = st \vert m ( w , {θ}_{st} ) ) \\ {ℒ}_{win} ( w ) = - log P ( {\tilde{y}}_{win} = {y}_{win} \vert w ) \\ ℒ = {ℒ}_{para} + α \cdot {ℒ}_{win} ( w ) + β \cdot {ℒ}_{st} ( w , {θ}_{st} ) + {ℒ}_{AL} ( {Z}_{M 2} , {Z}_{M 1} ) \\ y = Sigmoid ( δ * {\tilde{y}}_{M 1} + γ * {\tilde{y}}_{M 2} ) \\ {ϕ}_{i} ( {f}_{Z} , y ) \approx {m}_{{y}_{i} {f}_{Z}} ( {y}_{i} - E [ {y}_{i} ] ) \\ ϕ ( {f}_{Z} , {y}_{i} ) = \frac{1}{\vert E \vert} \frac{1}{\vert A \vert} {\sum }_{{x}_{M 1}^{′} ∈ E} {\sum }_{{x}_{M 2}^{′} ∈ A} ϕ ( {f}_{Z} , {x}_{M 1}^{′} , {x}_{M 2}^{′} , {y}_{i} )$$

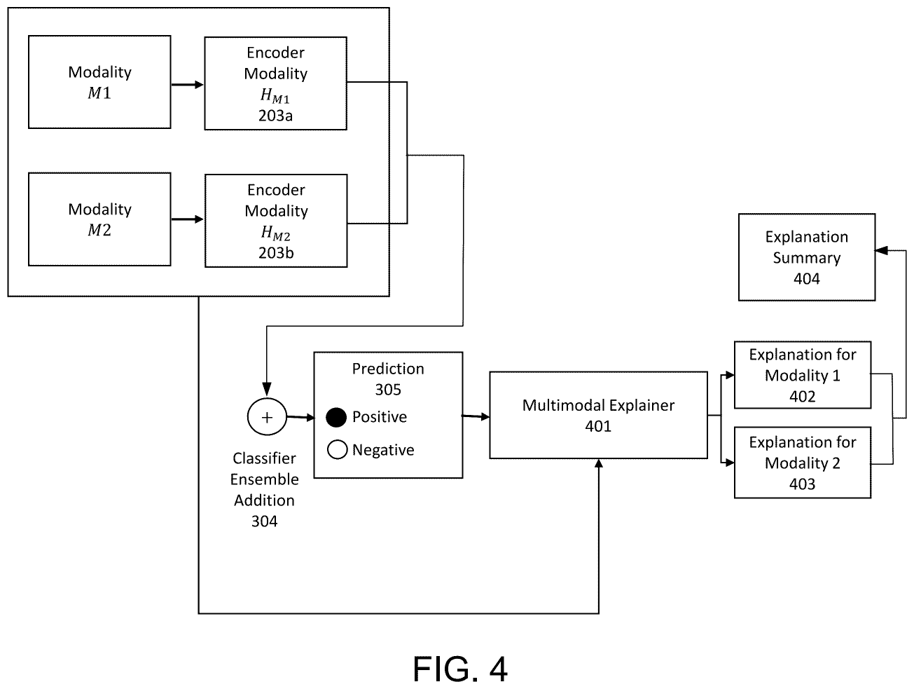

In FIG. 4, the correlations between the modalities that lead to the predicted outcome are assessed using a multimodal explainer module 401. Referring back to FIG. 3, a distribution of the input modality datasets M1, M2 are processed via an encoder network 203a, 203b, and the information from the multiple modalities can be correlated using a classifier ensemble addition of individual logits (via module 304), where a final classifier prediction module 305 outputs a positive or negative outcome (e.g., a prediction of a cognitive state or disorder of a subject). In various embodiments, the multimodal explainer module 401 can generate an explanation map for individual modalities 402, 403 to find the dependencies between the highest attributing features from all the modalities used in SMDL algorithm. Further, the multimodal explainer module can generate and output a summary 404 of the explanation maps for modalities with negative and positive correlations towards the final classifier predictions.

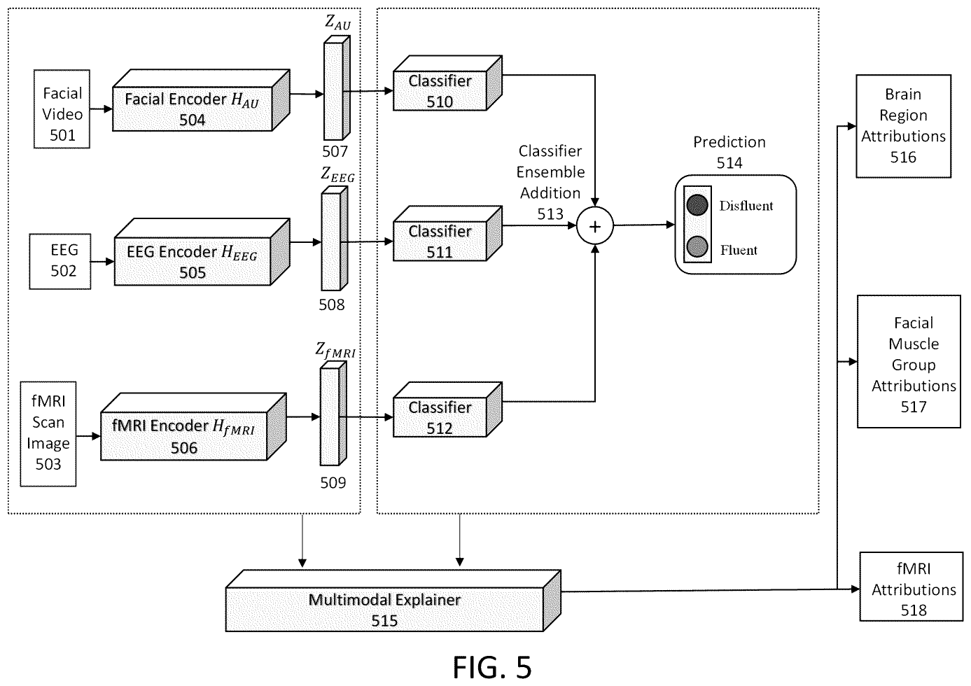

Next, FIG. 5 shows an exemplary the SMDL framework for predicting speech disfluency in an example scenario. Here, data from different modalities like facial video recordings 501, EEG 502, and fMRI scan images 503 are obtained as input data for a trained encoder network 203 having facial encoder 504, EEG encoder 505, and fMRI encoder 506. The modality encoders 504-506 create dense representations of corresponding EEG, Action Unit (AU) and fMRI inputs which are influenced by each other. The latent representations from pre-training of the individual deep learning models 507-509 are forced to align to each other using the alignment loss during the optimization. The distribution of EEG, AU and fMRI are made closer with an exemplary LDSDA algorithm. These distributions are passed to individual classifiers 510-512) and each output embeddings (Z_{EEG}, Z_{AU}, Z_{fMRI}) are combined with a weighted ensemble by a classifier ensemble addition module 513 and then the final prediction on whether the subject is fluent or disfluent is made by a prediction module 514. To explain the cognitive states and facial muscle movements with highest correlations to speech disfluency events, with multimodal explainer 515, an explanation map 516-518 each for individual modality (EEG, AU and fMRI) is generated to find dependencies between highest attributing features from each modalities. Accordingly, the multimodal explainer 515 can generate and output a summary containing the positive and negative correlations of EEG, AU and fMRI towards the final decision made by the prediction module 514.

In brief, domain specific artificial intelligence (AI) algorithms are highly performant in their respective domains. However, supervised AI algorithms require a manually labeled dataset which increase the cost, time, and human effort associated with training AI algorithms. These complexities increase considerably when there are multiple modalities of input data feeding AI models. In the present disclosure, systems and methods are provided for using machine learning (ML) algorithms to learn a source domain distribution from a large amount of multimodal unlabeled data. An exemplary source modality latent domain learning (SMDL) algorithm of the present disclosure can transfer the knowledge of source domain distribution to train the ML algorithm on a small amount of labeled target domain data. The domain distributions of individual modalities can be further aligned with each other by applying an alignment loss, such that a classifier for each modality takes the aligned distributions as input for performing a specific task. Such systems and methods for making diagnostic disease predictions based on multiple input sources are well-suited for domains like healthcare, since acquiring large amount of labeled can be very challenging.

Accordingly, disclosed systems and methods can be used for a variety of applications and scenarios. As an illustrative example, an exemplary machine learning method for multimodal domain adaptation and latent domain transfer to predict cognitive states and disorders of individuals comprises preparing multimodal input data of the following inputs including, but not limited to, streaming video of individual, EEG signal captured using wearable EEG caps, etc.; extracting time synchronized features from all modalities (e.g., extracting facial features from facial videos or extracting EEG signals from raw EEG data); training a multimodal machine learning algorithm on a small amount (“few-shot”) of input labeled data by learning multimodal signal correlations and source latent distribution alignment; saving the multimodal trained machine learning model; and/or loading the multimodal machine learning algorithm to system memory to predict events, including but not limited to, cognitive states, neurological disorders, and more.

In various exemplary embodiments, an exemplary multimodal machine learning method can learn multimodal correlations of source modalities from limited data by selecting a region-of-interest (ROI) from the individual source modalities and applying a signal transformation; learning individual source latent representations by learning to predict the ROI and signal transformations; aligning the distribution of individual source latent representations using alignment loss functions (which helps correlate individual modalities) to form aligned multimodal source latent vectors; and/or training a downstream machine learning prediction neural network using the aligned multimodal source latent vectors.

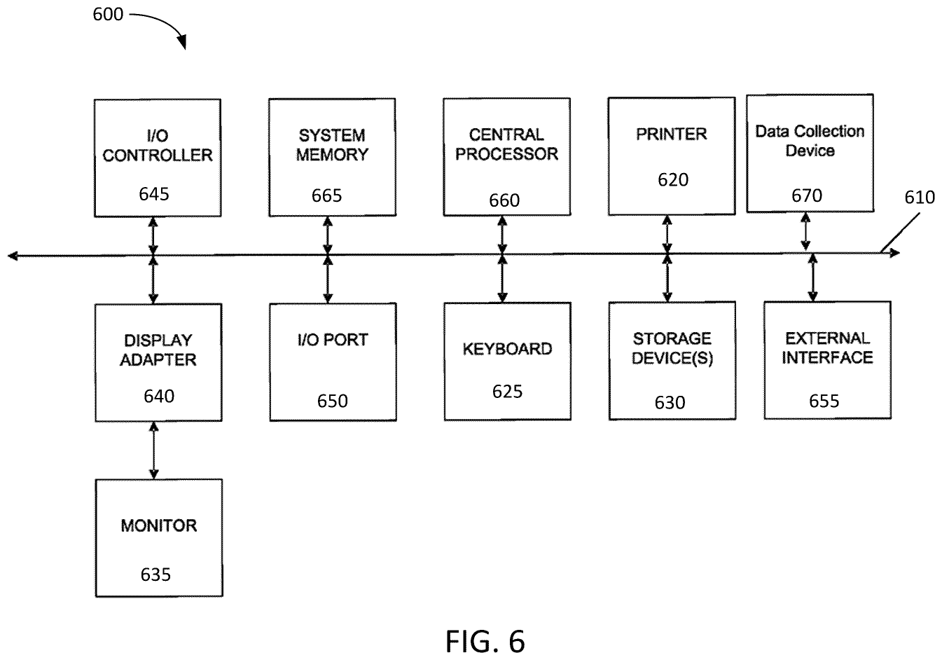

Various computer systems may utilize and/or execute the disclosed modules, frameworks, and processes disclosed herein. In some embodiments, as shown in FIG. 6, a computer system 600 includes a single computer apparatus, where the modules/frameworks/processes can be the components of the computer apparatus. In other embodiments, a computer system can include multiple computer apparatuses, each being a module or framework, with internal components. A computer system can include desktop and laptop computers, tablets, mobile phones and other mobile devices.

Exemplary modules, frameworks, and/or processes that can be implemented, stored, and/or executed in a computer system include, but are not limited to, input data module 101, source modality latent domain learning module 102, source modality signal transformer classifier module 103, source modality region of interest (ROI) classifier module 104, ROI selection module 201, signal transformation module 202, encoder for modality module 203, latent representation 204, input modalities (e.g., facial video recordings 501, EEG recordings 502, fMRI scan images 503), alignment loss functions, classifier modalities 302, 303, classifier ensemble computations 304, predictions 305, 514, multimodal explainer module 401, 515, output explanations 402, 304, explanation summaries 404, encoders 506-506, attributions 516-518, etc.

The components shown in FIG. 6 are interconnected via a system bus 610. Additional subsystems such as a printer 620, keyboard 625, storage device(s) 630, monitor 635 (e.g., a display screen, such as an LED), which is coupled to display adapter 640, and others are shown. Peripherals and input/output (I/O) devices, which couple to I/O controller 645, can be connected to the computer system 600 by any number of means known in the art such as input/output (I/O) port 650 (e.g., USB, FireWire®). For example, I/O port 650 or external interface 655 (e.g. Ethernet, Wi-Fi, etc.) can be used to connect computer system 600 to a wide area network such as the Internet, a mouse input device, or a scanner. The interconnection via system bus 610 allows a central processor 660 to communicate with each subsystem and to control the execution of a plurality of instructions from system memory 665 or the storage device(s) 630 (e.g., a fixed disk, such as a hard drive, or optical disk), as well as the exchange of information between subsystems. The system memory 665 and/or the storage device(s) 630 may embody a computer readable medium. Another subsystem is a data collection device 670, such as a camera, microphone, accelerometer, and the like. Any of the data mentioned herein can be output from one component to another component and can be output to the user.

According to an exemplary embodiment, the central processor 660 is a hardware device for executing software, particularly that stored in memory 665. The processor 660 can be any custom made or commercially available processor, a central processing unit (CPU), an auxiliary processor among several processors associated with the display adapter 640, a semiconductor based microprocessor (in the form of a microchip or chip set), a macroprocessor, or generally any device for executing software instructions. The memory 665 provides storage of instructions and data for programs executing on processor 660, such as one or more of the functions and/or modules disclosed herein. Also stored in the memory 665 may be a data store and other data, which stores information relevant to the disclosed systems and processes of the present disclosure, neural network models, AI or machine learning algorithms, etc. The data store can be located in a single installation or can be distributed among many different geographical or network locations. In various embodiments, an application programming interface (API) component operative on the system 600 may be provided to load, update, and serve machine learning models on the different computer platforms that interface with the computer system 600.

Additional processors may be provided, such as a graphics processing unit (GPU), an auxiliary processor to manage input/output, an auxiliary processor to perform floating-point mathematical operations, a special-purpose microprocessor having an architecture suitable for fast execution of signal-processing algorithms (e.g., digital-signal processor), a slave processor subordinate to the main processing system (e.g., back-end processor), an additional microprocessor or controller for dual or multiple processor systems, and/or a coprocessor. Such auxiliary processors may be discrete processors or may be integrated with processor 660.

A computer system can include a plurality of the same components or subsystems, e.g., connected together by external interface 655, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

It should be understood that the described processes may be embodied in one or more software modules that are executed by one or more hardware processors (e.g., processor 660), for example, as the application discussed herein. The described processes may be implemented as instructions represented in source code, object code, and/or machine code. These instructions may be executed directly by hardware processor(s) 660, or alternatively, may be executed by a virtual machine operating between the object code and hardware processors 660. In addition, the disclosed application may be built upon or interfaced with one or more existing systems.

Alternatively, the described processes may be implemented as a hardware component (e.g., general-purpose processor, integrated circuit (IC), application-specific integrated circuit (ASIC), digital signal processor (DSP), field-programmable gate array (FPGA) or other programmable logic device, discrete gate or transistor logic, etc.), combination of hardware components, or combination of hardware and software components. To clearly illustrate the interchangeability of hardware and software, various illustrative components, blocks, modules, circuits, and steps are described herein generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the claims included herein. In addition, the grouping of functions within a component, block, module, circuit, or step is for ease of description. Specific functions or steps can be moved from one component, block, module, circuit, or step to another.

Furthermore, while the processes, described herein, are illustrated with a certain arrangement and ordering of subprocesses, each process may be implemented with fewer, more, or different subprocesses and a different arrangement and/or ordering of subprocesses. In addition, it should be understood that any subprocess, which does not depend on the completion of another subprocess, may be executed before, after, or in parallel with that other independent subprocess, even if the subprocesses are described or illustrated in a particular order.

Certain embodiments of the present disclosure can be implemented in hardware, software, firmware, or a combination thereof. In various embodiments, such software or firmware is stored in computer-readable medium (e.g., a memory) and that is executed by a suitable instruction execution system. In various embodiments, such hardware can be implemented with any or a combination of the following technologies, which are all well known in the art: discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

In the context of this document, a “computer-readable medium” can be any means that can contain, store, communicate, or transport the program for use by or in connection with the instruction execution system, apparatus, or device. The computer readable medium can be, for example but not limited to, an electronic, magnetic, optical, electromagnetic, infrared, or semiconductor system, apparatus, or device. More specific examples (a nonexhaustive list) of the computer-readable medium would include the following: an electrical connection (electronic) having one or more wires, a portable computer diskette or drive (magnetic), a random access memory (RAM) (electronic), a read-only memory (ROM) (electronic), an erasable programmable read-only memory (EPROM or Flash memory) (electronic), an optical fiber (optical), and a portable compact disc read-only memory (CDROM) (optical).

The above description of the disclosed embodiments is provided to enable any person skilled in the art to make or use the invention. Various modifications to these embodiments will be readily apparent to those skilled in the art, and the general principles described herein can be applied to other embodiments without departing from the spirit or scope of the present disclosure. Thus, it is to be understood that the description and drawings presented herein represent various embodiments of the present disclosure and are therefore representative of the subject matter which is broadly contemplated by the present disclosure. It is further understood that the scope of the present disclosure fully encompasses other embodiments that may become obvious to those skilled in the art and that the scope of the present disclosure is accordingly not limited.

We claim:

1. A computer-implemented method comprising:
obtaining, by a computer system, multimodal input data of a subject, wherein the multimodal input data comprises at least two input modalities of data;
extracting, by the computer system, features from the multimodal input data;
learning, by the computer system, multimodal signal correlations and source latent distribution alignment from the extracted features of the multimodal input data;
training and optimizing, by the computer system, a multimodal machine learning algorithm on input labeled data to learn local features of each modality of the multimodal input data; and
executing, by the computer system, the trained multimodal machine learning algorithm to predict a cognitive state or disorder of the subject using the learned multimodal signal correlations and source latent distribution alignment.

2. The computer-implemented method of claim 1, wherein the multimodal input data comprises a video recording of the subject and an Electroencephalogram (EEG) recording of the subject.

3. The computer-implemented method of claim 2, wherein the features extracted from the video recording of the subject comprises facial features of the subject and the features extracted from the EEG recording comprises EEG signals.

4. The computer-implemented method of claim 2, wherein the multimodal input data further comprises a functional Magnetic Resonance Imaging (fMRI) recording.

5. The computer-implemented method of claim 1, wherein the learning the multimodal signal correlations and source latent distribution alignment from the extracted features of the multimodal input data comprises:
selecting a region-of-interest (ROI) from the individual source modalities and applying a signal transformation;
learning individual source latent representations by predicting the ROI and signal transformations;
aligning a distribution of individual source latent representations using alignment loss functions; and
training a machine learning prediction neural network using the aligned distribution of individual source latent representations.

6. The computer-implemented method of claim 1, further comprising:
generating, by the computer system, an explanation map for each input modality of data to explain the predicted cognitive state or disorder of the subject with respect to the extracted features from the multimodal input data; and
outputting, by the computer system, the explanation map.

7. The computer-implemented method of claim 6, further comprising:
generating, by the computer system, a summary of the explanation maps for each input modality of data with negative and positive correlations towards the predicted cognitive state or disorder of the subject; and
outputting, by the computer system, the summary of the explanation maps.

8. A system comprising:
at least one hardware processor; and
one or more software modules that are configured to, when executed by the at least one hardware processor, to:
obtain multimodal input data of a subject, wherein the multimodal input data comprises at least two input modalities of data;
extract features from the multimodal input data;
learn multimodal signal correlations and source latent distribution alignment from the extracted features of the multimodal input data;
train and optimize a multimodal machine learning algorithm on input labeled data to learn local features of each modality of the multimodal input data; and
execute the trained multimodal machine learning algorithm to predict a cognitive state or disorder of the subject using the learned multimodal signal correlations and source latent distribution alignment.

9. The system of claim 8, wherein the multimodal input data comprises a video recording of the subject and an Electroencephalogram (EEG) recording of the subject.

10. The system of claim 9, wherein the features extracted from the video recording of the subject comprises facial features of the subject and the features extracted from the EEG recording comprises EEG signals.

11. The system of claim 9, wherein the multimodal input data further comprises a functional Magnetic Resonance Imaging (fMRI) recording.

12. The system of claim 8, wherein the learning the multimodal signal correlations and source latent distribution alignment from the extracted features of the multimodal input data comprises:
selecting a region-of-interest (ROI) from the individual source modalities and applying a signal transformation;
learning individual source latent representations by predicting the ROI and signal transformations;
aligning a distribution of individual source latent representations using alignment loss functions; and
training a machine learning prediction neural network using the aligned distribution of individual source latent representations.

13. The system of claim 8, wherein the one or more software modules are configured to, when executed by the at least one hardware processor, to:
generate an explanation map for each input modality of data to explain the predicted cognitive state or disorder of the subject with respect to the extracted features from the multimodal input data; and
output the explanation map.

14. The system of claim 13, wherein the one or more software modules are configured to, when executed by the at least one hardware processor, to
generate a summary of the explanation maps for each input modality of data with negative and positive correlations towards the predicted cognitive state or disorder of the subject; and
output the summary of the explanation maps.

15. A non-transitory computer-readable medium having instructions stored therein, wherein the instructions, when executed by a processor, cause the processor to:
obtain multimodal input data of a subject, wherein the multimodal input data comprises at least two input modalities of data;
extract features from the multimodal input data;
learn multimodal signal correlations and source latent distribution alignment from the extracted features of the multimodal input data;
train and optimize a multimodal machine learning algorithm on input labeled data to learn local features of each modality of the multimodal input data; and
execute the trained multimodal machine learning algorithm to predict a cognitive state or disorder of the subject using the learned multimodal signal correlations and source latent distribution alignment.

16. The non-transitory computer-readable medium of claim 15, wherein the multimodal input data comprises a video recording of the subject and an Electroencephalogram (EEG) recording of the subject.

17. The non-transitory computer-readable medium of claim 16, wherein the features extracted from the video recording of the subject comprises facial features of the subject and the features extracted from the EEG recording comprises EEG signals.

18. The non-transitory computer-readable medium of claim 16, wherein the multimodal input data further comprises a functional Magnetic Resonance Imaging (fMRI) recording.

19. The non-transitory computer-readable medium of claim 15, wherein the learning the multimodal signal correlations and source latent distribution alignment from the extracted features of the multimodal input data comprises:
selecting a region-of-interest (ROI) from the individual source modalities and applying a signal transformation;
learning individual source latent representations by predicting the ROI and signal transformations;
aligning a distribution of individual source latent representations using alignment loss functions; and
training a machine learning prediction neural network using the aligned distribution of individual source latent representations.

20. The non-transitory computer-readable medium of claim 15, wherein the instructions, when executed by a processor, cause the processor to:
generate an explanation map for each input modality of data to explain the predicted cognitive state or disorder of the subject with respect to the extracted features from the multimodal input data;
output the explanation map;
generate a summary of the explanation maps for each input modality of data with negative and positive correlations towards the predicted cognitive state or disorder of the subject; and
output the summary of the explanation maps.
